(19) United States
(12) Patent Application Publication
(10) Pub. No.: US 2017/0203105 A1
(43) Pub. Date: Jul. 20, 2017

(54) Feedthrough Of A Medical Electronic Device, Method For Producing Same, And Medical Electronic Device

(71) Applicant: BIOTRONIK SE & Co. KG, Berlin (DE)

(72) Inventors: Thomas Sontheimer, Rosstal (DE); Kathrin Zecho, Wiesenthau (DE); Josef Teske, Hallstadt (DE)

(21) Appl. No.: 15/389,547

(22) Filed: Dec. 23, 2016

(30) Foreign Application Priority Data
Jan. 20, 2016 (DE) 10 2016 100 865.6

(51) Int. Cl. A61N 1/375; H01R 13/52; A61N 1/39; H01R 13/523; A61N 1/36; A61N 1/362

(52) U.S. Cl. CPC A61N 1/3754; A61N 1/36032; A61N 1/362; A61N 1/3956; H01R 13/523; H01R 13/5202; H01R 13/5216

(57) ABSTRACT

A feedthrough of a medical electronic device, which in particular is implantable, including a housing and at least one electrical or electronic component received in the housing, wherein the feedthrough has a feedthrough flange for closing an opening in the housing and for receiving a multiplicity of connection elements in an insulating body surrounding the connection elements, which connection elements serve for the connection of a component or at least one component, externally of the housing, wherein the insulating body is formed from multi-layer ceramic, in particular from HTCC, with sintered-in pre-configured conductor elements, and comprising a device-specific contact-making means of selected conductor elements, which together with the contacted pre-configured conductor elements forms the connection elements.

### CROSS-REFERENCE TO RELATED APPLICATIONS

This patent application claims the benefit of and priority to co-pending German Patent Application No. DE 10 2016 100 865.6, filed on Jan. 20, 2016 in the German Patent Office, which is hereby incorporated by reference in its entirety.

### TECHNICAL FIELD

The present invention relates to a feedthrough of an implantable medical electronic device and also to a device of this type. This device typically comprises a device housing, in which electronic and electrical function units are housed, and the feedthrough has a feedthrough flange for closing an opening in the housing and for receiving a multiplicity of connection elements in an insulating body surrounding the connection elements, which connection elements serve for the connection of a component or at least one component, externally of the housing. The present invention also relates to a method for producing such a feedthrough

### BACKGROUND

Implantable devices of the above-mentioned type have long been used on a mass scale, in particular as cardiac pacemakers or implantable cardioverters (especially defibrillators). However, said device may also be a less complex device, such as an electrode line or sensor line or also a cochlear implant.

Most implantable medical electronic devices of practical significance are intended to deliver electrical pulses to excitable body tissue via suitably placed electrodes. In order to perform this function, electronic/electrical function units for generating the pulses and for suitably controlling the pulse generation are housed in the housing of the device, and electrodes or connections are provided directly on the device externally for at least one electrode line, in the distal end portion of which the electrodes are attached to the tissue for pulse transmission.

The electronic/electrical function units in the device interior are to be connected to the outer electrodes or electrode line connections in such a way that ensures utterly and permanently reliable function under the special conditions of the implanted state. Furthermore, the feedthrough of such a device has to ensure that said device is sealed permanently under these conditions.

In particular, feedthroughs of which the main and insulating body consists substantially of ceramic or glass are known, wherein multilayer or multi-part superstructures have also been developed with use of metals or metal oxides and are used. Such known feedthroughs largely satisfy the requirements placed thereon.

A feedthrough flange can be produced by eroding material processing (milling) or alternatively by means of an MIM (metal injection moulding) method. In this method, a mixture of metal powder and organic binder is injected into a mold, demolded and then sintered. In this method, the flange is produced as a whole in one step.

With regard to the production of the feedthrough on the whole, this is currently typically joined in a number of processes from a relatively large number of individual components (insulation ceramic, feedthrough flange, connection element, solder, etc.), wherein some of the components pass through upstream thermal and shaping processes, such that the production of a feedthrough comprises a large number of process steps, some of which are to be carried out at high temperatures. These methods involve a lot of work and energy and in addition are relatively time-consuming and therefore are costly on the whole.

The present invention is directed toward overcoming one or more of the above-mentioned problems.

### SUMMARY

An object of the present invention is to specify an improved feedthrough of the type specified in the introduction, which in particular can be produced in a simplified production process with increased energy efficiency and thus more economically and provides more degrees of freedom in respect of the construction. Furthermore, a corresponding production method will be specified and an implantable medical electronic device which can be produced relatively easily and economically will be provided.

At least this object is achieved in terms of its device aspects by a feedthrough having the features of claim 1 and by a medical electronic device having the features of claim 13. In terms of its method aspects, at least the object is achieved by methods having the features of claim 6. Expedient developments are specified in the respective dependent claims.

The present invention includes the concept of modifying the conventional structure of a feedthrough such that there are in principle a smaller number of individual components and a smaller number of individual process steps. In particular, in accordance with a further concept of the present invention, the number of subsequent joining steps can be reduced by partial structural combining of individual components within the scope of upstream process steps. In accordance with yet a further concept of the present invention, this concerns the structure of the insulation body and connection elements or parts hereof as an integral preliminary product. As a result of subsequent thermal treatment of this integral preliminary product, traditionally separate thermal process steps for insulation ceramic and connection elements can and will be combined.

Provision is made in accordance with the present invention so that the insulating body is formed from multi-layer ceramic with sintered-in pre-configured conductor elements and is provided with a device-specific contact-making means of selected conductor elements, which together with the contacted preconfigured conductor elements forms the connection elements. The insulating body is particularly a high-temperature multi-layer ceramic (HTCC), the technology of which has become established in the meantime in the production of feedthroughs for medical electronic devices and can be adapted without difficulty to the present invention.

In an embodiment of the present invention, the insulating body is formed as a fragment of a pre-fabricated insulating body array. This embodiment enables an amalgamation of process steps for individual components to an even greater extent than the basic concept of the present invention and enables an associated saving of energy and process time. The pre-fabricated insulating body array is expediently configured with predetermined breaking points, which enable a simple separation, in particular without tools, of the individual insulating bodies; this embodiment, however, is not limited hereto.

In a further embodiment, provision is made so that the feedthrough flange is a metal powder injection molded part which is injected around the insulating body and is shrink fitted thereon. The concept of the present invention is consequently also further detailed hereby in that the separate pre-manufacture of a feedthrough flange and a separate step of the joining of insulating body and flange are spared and the corresponding handling and assembly outlay can be spared.

In a further embodiment, the device-specific contact-making means comprises thick-film contact elements in the form of known structures of thick-film or hybrid electronics. In this embodiment, known and tried and tested construction and method elements of thick-film electronics can be used advantageously without the need for new structural or technical developments and corresponding testing and functional substantiation.

In a first embodiment of the concept of the present invention, the device-specific contact-making means comprises separate contact elements, in particular connection pins, applied selectively to the pre-configured conductor elements. Separate components are again used here, but these can be attached to the insulating body provided with the device-specific contact-making means in process sequences that can be advantageously integrated, without significantly increasing the complexity of the production method. The embodiment additionally enables a realization of the inventive concept in conjunction with largely conventional connection geometries of the medical electronic device, without significant structural alteration thereof.

In a further embodiment, the pre-configured conductor elements or some thereof provide a filter function of the feedthrough, for example in the form of a conventional filter capacitor associated with the connection pins. In an embodiment, this function is provided with incorporation of elements of the selectively applied device-specific contact-making means.

From method aspects, the present invention includes the concept that the insulating body is formed with the pre-configured conductor elements in an HTCC method and is then provided with the device-specific contact-making means and is surrounded by the feedthrough flange. Provision is especially made here so that the insulating body in the dried or “green” state is introduced into an injection mold adapted to the form of the feedthrough flange and is overmolded by the flange portion by injection of metal into the injection mold, and the composite, pre-fabricated in this way, formed of insulating body and feedthrough flange is then debound as a whole and sintered. A material, in particular titanium, which compared to the material of the insulating body, in particular aluminum oxide, has a higher coefficient of thermal expansion, is for this purpose selected especially as material for the feedthrough flange, whereby the feedthrough flange is thus shrink fitted onto the insulating body during the sintering. If titanium is selected as feedthrough flange material, sintering is preferably performed with oxygen exclusion (in a vacuum or in hydrogen) in order to counteract oxidation caused by the high oxygen affinity of titanium. So as to, at the same time, eliminate an undesirable discontinuous grain growth with formation of very large crystals in the aluminum oxide, a doping with MgO is provided in particular. However, the embodiment is not limited to the specified materials, but can also be implemented with other material combinations which meet the specified condition for the coefficient of thermal expansion.

The production of device-specific contact-making means is possible expediently in various variants: on the one hand, it can be performed once the insulating body has been surrounded by the feedthrough flange; on the other hand, a thick-film contacting step (for example with a mask structure or by selective layer application) can be performed. Lastly, for the device-specific contacting of selected conductor elements, an equipping with individual contact elements, in particular by having these soldered on, is provided in a further embodiment. All specified steps or techniques are known per se from the field of ceramic thick-film or hybrid circuits, and thus there is no need for a more detailed description here.

Further embodiments, features, aspects, objects, advantages, and possible applications of the present invention could be learned from the following description, in combination with the Figures, and the appended claims.

### BRIEF DESCRIPTION OF THE DRAWINGS

Advantages and expedient features of the present invention will also become clear from the description of exemplary embodiments with reference to the drawings, in which:

FIG. 1 shows a schematic, partially sectional illustration of an implantable medical electronic device.

FIG. 2 shows a schematic illustration, in part as longitudinal sectional illustrations, of the production sequence and structure of an embodiment of the feedthrough according to the present invention.

### DETAILED DESCRIPTION

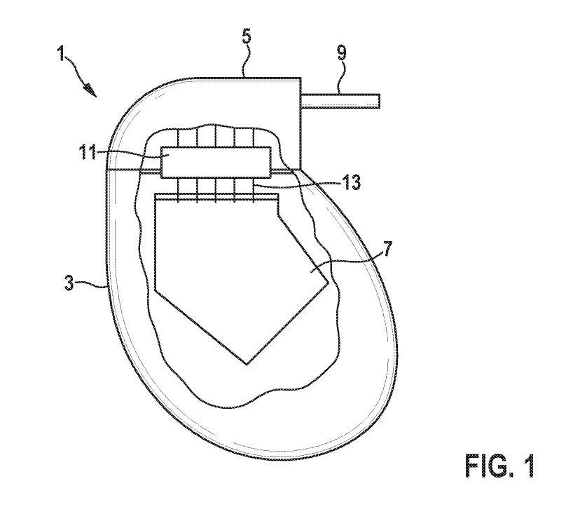

FIG. 1 shows a cardiac pacemaker 1 with a pacemaker housing 3 and a head part (header) 5, in the interior of which a printed circuit board (PCB) 7 is arranged, in addition to other electronic components, with an electrode line 9 being connected to the line connection (not shown) of said pacemaker 1 arranged in the header 5. A feedthrough 11 provided between the device housing 3 and header 5 comprises a multiplicity of connection elements 13. The connection pins 13 are inserted at one end through a corresponding bore in the printed circuit board 7 and are soft-soldered thereto.

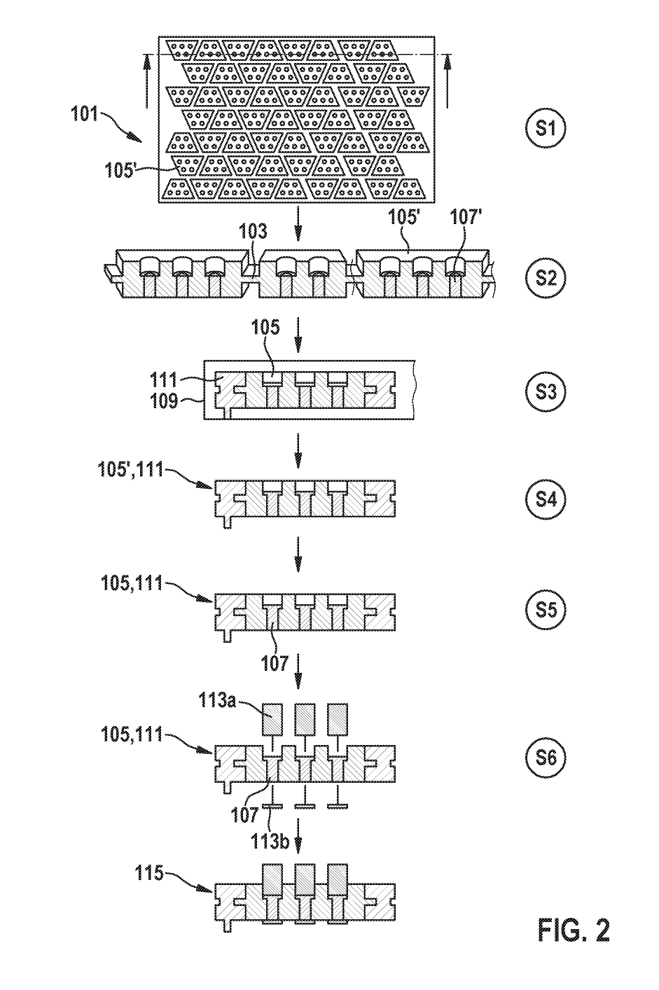

FIG. 2 schematically shows, in the form of a flow diagram associated with cross-sectional illustrations of the feedthrough 11, on the one hand the basic structure of an exemplary embodiment of the feedthrough according to the present invention and on the other hand key steps of the production of this feedthrough. It should be noted that the illustration is purely schematic and that neither details nor sizes of individual parts are intended to be correctly reproduced. It should also be noted that the sequence of steps of the production method has in no way been shown here in full.

Firstly, in a step S1, an insulating body array 101 formed from a multiplicity of regularly arranged raw insulating bodies 105′ connected to one another at predetermined breaking points 103 and having embedded conductor element precursors 107′ is produced by means of HTCC technology, which is known per se. The base is an aluminum oxide ceramic, and a material known per se from hybrid technology for conductive tracks, for example, based on silver or copper, can be used for the embedded conductor elements.

Still in the “green” state, the insulating body 105 is separated from the insulating array 101 in a step S2 by breaking at the predetermined breaking points 103. In a subsequent step S3, the separated raw insulating body 105′ is placed in a metal injection mold 109, in which a mold cavity in the form of a feedthrough flange is provided, and is over molded by means of MIM technology by titanium, which in the mold cavity forms a feedthrough flange 111 surrounding the raw insulating body 105′. In a further step S4, the pre-fabricated integral component constituted by the insulating body/flange 105′/111 is debound as a whole, and in a further step S5 a common sintering operation is performed at temperatures T>1300° C., preferably in reducing atmosphere, wherein a shrink fitting onto the insulating body portion consisting of Al₂O₃ occurs as a result of the slightly higher coefficient of thermal expansion of the flange material (titanium). At the same time, the finished insulating body 105, with completely conductive metal conductor elements 107, is produced from the raw insulating body 105′.

In an optional, further step S6, a device-specific contact-making means is applied to the integral insulating body/flange 105/111, which is finished as such in step S5. This is symbolized in the Figure by application on both sides of first and second contact elements 113a, 113b to all conductor elements 107; however, neither of the two-sided additional contacting nor a contacting of all pre-fabricated, embedded conductor elements is compulsory. The attachment can be implemented, for example, by soldering or bonding or coating, for example, with an Ag colloid. Should a subsequent soldering with Nb connection pins be necessary, the HTCC insulating body could be pre-conditioned by means of an active solder (for example Ti Cu Ni). On the whole, a feedthrough 115 is provided which is configured so as to be suitable for the production of the necessary electrical connections of a specific medical electronic device and which has been produced in a production process of simplified sequence, with reduced handling, outlay and lower energy consumption.

The present invention can also be embodied in a multiplicity of modifications of the examples shown here and aspects of the present invention detailed further above.

It will also be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range.

1. A feedthrough of a medical electronic device, which is implantable, comprising:
a housing; and
at least one electrical or electronic component received in the housing, wherein the feedthrough has a feedthrough flange for closing an opening in the housing and for receiving a multiplicity of connection elements in an insulating body surrounding the connection elements, which connection elements serve for the connection of a component or at least one component, externally of the housing, wherein the insulating body is formed from multi-layer ceramic, in particular from HTCC, with sintered-in pre-configured conductor elements, and comprising a device-specific contact-making means of selected conductor elements, which together with the contacted pre-configured conductor elements forms the connection elements.

2. The feedthrough according to claim 1, wherein the insulating body is formed as a fragment of an insulating body array.

3. The feedthrough according to claim 1, wherein the feedthrough flange is a metal powder injection-molded part, which is injected around the insulating body and is shrink fitted thereon.

4. The feedthrough according to claim 1, wherein the pre-fabricated conductor elements and/or the device-specific contact-making means comprise/comprises thick-film contact elements.

5. The feedthrough according to claim 1, wherein the device-specific contact-making means comprises separate contact elements, in particular connection pins, which are applied selectively to the pre-configured conductor elements.

6. The feedthrough according to claim 1, wherein the pre-configured conductor elements, optionally in conjunction with the device-specific contact-making means, provide a filter device of the feedthrough.

7. A method for producing a feedthrough according to claim 1, wherein the insulating body is formed with the pre-configured conductor elements in an HTCC method and is then provided with the device-specific contact-making means and is surrounded by the feedthrough flange.

8. The method according to claim 7, wherein the insulating body is formed as part of an insulating body array and is then separated.

9. The method according to claim 7, wherein the insulating body in the dried or “green” state is introduced into an injection mold adapted to the form of the feedthrough flange and is overmolded by the feedthrough flange by injection of metal into the injection mold, and the composite, thus pre-fabricated, formed of raw insulating body and feedthrough flange is then debound as a whole and sintered.

10. The method according to claim 9, wherein a material, in particular titanium, which has a higher coefficient of thermal expansion compared to the material of the insulating body, in particular aluminum oxide, is selected as material for the feedthrough flange, whereby the feedthrough flange is shrink fitted onto the insulating body during the sintering.

11. The method according to claim 10, wherein the feedthrough flange is formed from titanium and the insulating body is formed from aluminum oxide, wherein the raw insulating body and feedthrough flange are sintered as a whole with oxygen exclusion, and in particular the raw insulating body is doped with magnesium oxide.

12. The method according to claim 7, wherein the device-specific contacting of selected pre-configured conductor elements is performed once the insulating body has been surrounded by the feedthrough flange.

13. The method according to claim 7, wherein a thick-film contacting step is carried out for the device-specific contacting of selected conductor elements.

14. The method according to claim 7, wherein, for device-specific contacting of selected conductor elements, an equipping with individual contact elements, in particular by having these soldered on, is performed.

15. A medical electronic device comprising a feedthrough according to claim 1, in particular formed as a cardiac pacemaker, cardioverter, or cochlear implant.
